(19) United States
(12) Patent Application Publication
(10) Pub. No.: US 2022/0409054 A1
(43) Pub. Date: Dec. 29, 2022

(54) Resonant Circuit-Based Vascular Monitors and Related Systems and Methods

(71) Applicant: Foundry Innovation & Research 1, Ltd., Dublin (IE)

(72) Inventors: Pablo I. Martin, Dublin (IE); Michael Kelly, Dublin (IE); Friedrich Wetterling, Dublin (IE); Jack McDonald, Dublin (IE); Fiachra M. Sweeney, Dublin (IE)

(21) Appl. No.: 17/775,836

(22) PCT Filed: Nov. 12, 2020
(86) PCT No.: PCT/IB2020/060669

§ 371 Date: May 10, 2022

Related U.S. Application Data
(60) Provisional application No. 62/934,399, filed on Nov. 12, 2019.

(51) Int. Cl. A61B 5/00; A61B 5/02

(52) U.S. Cl. CPC A61B 5/0031; A61B 5/02007; A61B 5/6876; A61B 5/7203

(57) ABSTRACT

Systems and methods for control and signal processing in variable inductance, resonant circuit monitoring devices are disclosed, including improved techniques for energizing the sensor resonant circuit using excitation signal frequency sweeps, techniques for validating sensor readings and characterizing sensor frequency outputs to measured physical parameters and improved techniques for isolating background electromagnetic noise and distinguishing knows from sensor measurement signals.

### RELATED APPLICATIONS

The present application claims priority to U.S. Provisional Patent Application No. 62/934,399, filed Nov. 12, 2019, entitled “Resonant Circuit-Based Monitors and Related Systems and Methods,” which is incorporated by reference herein.

### FIELD

The present disclosure relates to improvements in wireless vascular monitors, in particular, resonant circuit-based monitors and related systems and methods.

### BACKGROUND

Resonant circuit (RC) based sensors are sensors that deliver a change in resonant frequency as a result of a change in a physical parameter in the surrounding environment, which change causes the resonant frequency produced by the circuit within the device to change. The change in resonant frequency, which may be detected as a “ring-back” signal when the circuit is energized, indicates the sensed parameter or change therein. As is well-known, a basic resonant circuit includes an inductance and a capacitance. In most available RC sensing devices, the change in resonant frequency results from a change in the capacitance of the circuit. The plates of a capacitor moving together or apart in response to changes in pressure, thus providing a pressure sensor, is a well-known example of such a device. Less commonly, the change in resonant frequency is based on a change in the inductance of the circuit.

The present Applicant has filed a number of patent applications disclosing new RC monitoring devices using variable inductance for monitoring intravascular dimensions and determining physiological parameters such as patient fluid state based thereon. See, for example, PCT/US17/63749, entitled “Wireless Resonant Circuit and Variable Inductance Vascular Implants for Monitoring Patient Vasculature and Fluid Status and Systems and Methods Employing Same”, filed Nov. 29, 2017 (Pub. No. WO2018/102435) and PCT/US19/34657, entitled “Wireless Resonant Circuit and Variable Inductance Vascular Monitoring Implants and Anchoring Structures Therefore”, filed May 30, 2019 (Pub. No. WO2019/232213), each of which is incorporated by reference herein, which disclose a number of different embodiments and techniques related to such devices.

Notwithstanding the advances in the art represented by these prior disclosures, improvements in control and signal processing for such devices can still be made. The present disclosure thus offers solutions to some unique problems described herein, which have been encountered only after introduction and testing of the aforementioned new RC monitoring devices.

### SUMMARY OF THE DISCLOSURE

In one implementation, the present disclosure is directed to a method for controlling a wireless, resonant circuit sensor, the sensor including a variable inductance coil that changes resonant frequency in response to a change in a monitored physical parameter and produces a ring-back signal at a frequency correlated to the physical parameter when energized. The method includes outputting at least one excitation frequency sweep comprising a preestablished number of transmit pulses at pre-defined frequencies over a range of expected implant resonant frequencies; receiving the ring-back signals for each of the sequentially output transmit pulses; transmitting at least one initial transmit pulse for a predetermined initial period, wherein the at least one initial transmit pulse comprises one of—a pulse frequency corresponding to the highest amplitude ring-back signal received from the at least one frequency sweep; or plural the excitation frequency sweeps; receiving plural test ring-back signals in response to at least one initial transmit pulse transmitted over the initial period; identifying an initial ring-back signal corresponding to a preferred excitation pulse frequency; and selecting the preferred excitation pulse frequency as a measurement transmit pulse frequency; outputting measurement transmit pulses at the measurement transmit pulse frequency for a subsequent measurement period.

In another implementation, the present disclosure is directed to a control system for a wireless, resonant circuit sensor, the sensor including a variable inductance coil that changes resonant frequency in response to a change in a monitored physical parameter and produces a ring-back signal at a frequency correlated to the physical parameter when energized. The control system includes a transmit/receive switch configured to control signal transmission to and signal receiving from an antenna, a signal generation module configured to generate excitation signals wherein the transmit receive switch controls transmission of the generated signal to the antenna, and a receiver-amplifier module configured to receive and process ring-back-signals received by the antenna and communicated to the receiver-amplifier module by the transmit/receive switch communicating with a processor configured to execute program instructions, characterized in that the system is configured to: output at least one excitation frequency sweep comprising a preestablished number of transmit pulses at pre-defined frequencies over a range of expected implant resonant frequencies; receive the ring-back signals for each of the sequentially output transmit pulses; transmit at least one initial transmit pulse for a predetermined initial period, wherein the at least one initial transmit pulse comprises one of—a pulse frequency corresponding to the highest amplitude ring-back signal received from the at least one frequency sweep; or plural the excitation frequency sweeps; receive plural test ring-back signals in response to at least one initial transmit pulse transmitted over the initial period; identify an initial ring-back signal corresponding to a preferred excitation pulse frequency; select the preferred excitation pulse frequency as a measurement transmit pulse frequency; and output measurement transmit pulses at the measurement transmit pulse frequency for a subsequent measurement period.

In still another implementation, the present disclosure is directed to a method for characterizing a resonant circuit sensor to correlate sensor output to a measured physical parameter, wherein the sensor comprises a variable inductance coil that changes resonant frequency in response to a change in the physical parameter by producing, when energized, a ring-back signal at a frequency correlateable to the physical parameter. The method includes determining physical parameter value versus frequency data over a range of parameter values and frequencies for at least one the sensor prior to placement in a patient; and creating a characterization curve for the at least one sensor by plotting a curve with the data using curve fitting or interpolation techniques.

In yet another implementation, the present disclosure is directed to a method for assessing electromagnetic background noise prior to outputting an excitation signal for conducting a measurement with a resonant circuit sensor, wherein the sensor comprises a variable inductance coil that changes resonant frequency in response to a change in a physical parameter by producing, when energized, a ring-back signal at a frequency correlateable to the physical parameter. The method includes transmitting predetermined a test pulse at a test frequency, wherein the test frequency is selected to be sufficiently distant from an expected sensor excitation frequency so as to not energize the sensor; receiving a test signal with a sensor ring-back signal receiver, wherein the received test signal is made up of the test pulse and background electromagnetic noise; defining the background electromagnetic noise based on the received test signal as signal components distinct from the known test pulse; and modulating signal processing of the received measurement ring-back signal to eliminate or reduce effects of the defined background electromagnetic noise.

In a further implementation, the present disclosure is directed to a method for validating a sensor signal in a resonant circuit sensor, wherein the sensor comprises a variable inductance coil that changes resonant frequency in response to a change in a physical parameter by producing, when energized, a ring-back signal at a frequency correlateable to the physical parameter. The method includes transmitting a known fixed frequency and fixed amplitude signal; capturing the known signal as a portion of a captured signal including a ring-back signal generated by the sensor; comparing the captured known signal portion with the transmitted known signal; and validating the sensor ring-back signal when the captured known signal portion matches the transmitted known signal within predetermined limits.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a schematic system overview of an embodiment of a wireless vascular monitoring system employing a resonant circuit-based sensor implant.

FIG. 2 is a block diagram of an embodiment of a control system for wireless vascular monitoring systems disclosed herein.

FIGS. 3A, 3B and 3C illustrate signals obtained in in vivo pre-clinical experiments using a prototype RC-WVM system as disclosed herein.

FIGS. 4A and 4B illustrate exemplary ring-back signals as received in bench top tests via a control system receiver-amplifier module without and with transmit to receive excitation signal leakage according to an embodiment disclosed herein.

FIG. 5 is an example of a sensor characterization curve.

### DETAILED DESCRIPTION

The unique physiology of the Inferior Vena Cava (IVC) presents some distinctive challenges in attempting to detect and interpret changes in its dimensions arising from changes in patient fluid state. For example, the IVC wall in a typical monitoring region (i.e., between the hepatic and renal veins) is relatively compliant compared to other vessels, which means that changes in vessel volume can result in different relative distance changes between the anterior-posterior walls as compared to the lateral-medial walls. Thus, it is quite typical that changes in fluid volume will lead to paradoxical changes in the geometry and motion of the vessel; that is, as the blood volume reduces the IVC tends to get smaller and collapse with respiration, and as the blood volume increases the IVC tends to get larger and the collapse with respiration is reduced. The present Applicant has developed new wireless sensor implants and related systems and methods in order to address these challenges and provide clinically effective wireless vascular monitors (“WVM”). In one such embodiment, the WVM comprises a resonant circuit configured as a coil implantable in the patient's vasculature (“RC-WVM”). Detailed examples of embodiments of RC-WVM, systems and methods are disclosed, inter alia, in Applicant's co-pending U.S. patent application Ser. No. 17/018,194, entitled “Wireless Resonant Circuit and Variable Inductance Vascular Monitoring Implants and Anchoring Structures Therefore”, filed Sep. 11, 2020, which is incorporated by reference herein in its entirety.

In the course of working with RC-WVM embodiments as described in the above-referenced application, Applicant has developed a number of new embodiments as disclosed herein that further improve accuracy and useability of RC-WVM implants, systems and methods as previously described. These new embodiments are described below after a basic overview discussion of one example of a RC-WVM system and its operation.

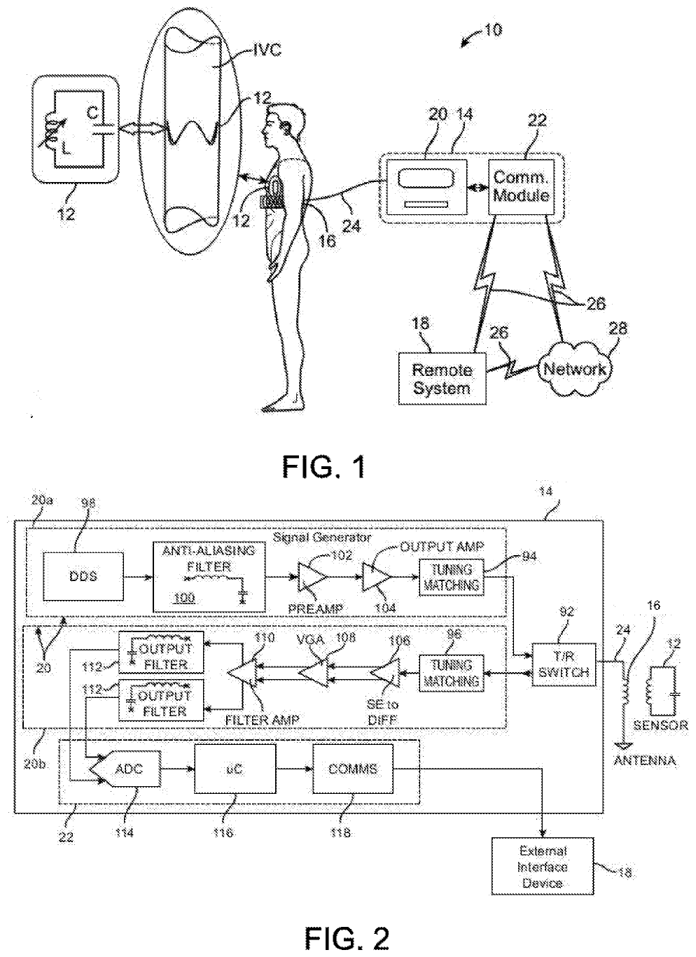

FIG. 1 provides an overview of an RC-WVM system 10 to which embodiments disclosed herein are applicable. As shown therein, such a system may generally comprise RC-WVM implant 12 configured for placement in a patient's inferior vena cava (IVC), control system 14, antenna module 16 and one or more remote systems 18 such as processing systems, user interface/displays, data storage, etc., communicating with the control and communications modules through one or more data links 26. Data links 26 may be wired or remote/wireless data links. In many implementations, remote system 18 may comprise a computing device and user interface, such as a laptop, tablet or smart phone, which serves as an external interface device.

RC-WVM implants 12 generally comprise a variable inductance, constant capacitance, resonant L-C circuit formed as a collapsible and expandable coil structure, which, when positioned at a monitoring position within the patient's IVC, moves with the IVC wall as it expands and contracts due to changes in fluid volume. The variable inductance is provided by the coil structure of the implant such that the inductance changes when the dimensions of the coil (e.g., the area surrounded by the coil or the “sensor area”) change with the IVC wall movement. The capacitive element of the circuit may be provided by a discrete capacitor or specifically designed inherent capacitance of the implant structure itself. When an excitation signal is directed at the RC-WVM implant, the resonant circuit produces a “ring-back” signal at a frequency that is characteristic of the circuit. The characteristic frequency changes based on changes in the size of the inductor, i.e. the coil, as it changes with the vessel wall. Because the inductance value is dependent on the geometry of the implant, which changes as mentioned above based on dimensional changes of the IVC in response to fluid state, heart rate etc., the ring-back signal can be interpreted by control system 14 to provide information as to the IVC geometry and therefore fluid state and other physiological information such as respiratory and cardiac rates.

Control system 14 comprises, for example, functional modules for signal generation, signal processing and power supply (generally comprising the excitation and feedback monitoring (“EFM”) circuits and indicated as module 20, comprising signal generation module 20a and receiver-amplifier module 20b as shown in FIG. 2) and communications and data acquisition module 22 to facilitate communication and data transfer to various external or remote systems 18 through data links 26 and optionally other local or cloud-based networks 28. After analyzing signals received from RC-WVM implant 12, results may be communicated manually or automatically through an external or remote system 18 to the patient, a caregiver, a medical professional, a health insurance company, and/or any other desired and authorized parties in any suitable fashion (e.g., verbally, by printing out a report, by sending a text message or e-mail, or otherwise). As shown in FIG. 2, components of control system 14 may comprise: transmit/receive (T/R) switch 92, transmitter tuning-matching circuit 94, receiver tuning-matching circuit 96, direct digital synthesizer (DDS) 98, anti-aliasing filter 100, preamplifier 102, output amplifier 104, single ended to differential input amplifier (SE to DIFF) 106, variable gain amplifier (VGA) 108, filter amplifier (e.g., an active band-pass filter-amplifier) 110, output filters (e.g., passive, high-order low pass filters) 112, high-speed analog-to-digital converter (ADC) 114, microcontroller 116, and communications sub-module 118. Signal identification, selection and other signal processing functions subsequent to amplification and filtering may be embedded within microcontroller 116 or may be executed in an external interface device 18 such as an external computing system execution program instructions for carrying out the steps disclosed herein.

Antenna module 16 is connected to control system 14 by power and communication link 24, which may be a wired or wireless connection. Antenna module 16 creates an appropriately shaped and oriented magnetic field around RC-WVM implant 12 based on signals provided by the signal generation module 20a of control system 14 in order to excite the resonant circuit as described above. Antenna module 16 thus provides both a receive function/antenna and a transmit function/antenna. In some embodiments the transmit and receive functionality are performed by a single antenna, which is switched between transmit and receive modes, for example by transmit/receive switch 92 (which may be a single pole, double throw switch). In other embodiments, each function is performed by a separate antenna.

As will be appreciated by persons skilled in the art, optimal excitation of an L-C resonant circuit occurs when the excitation signal is delivered at the circuit's natural frequency. However, in an RC-WVM implant 12 as described herein, the circuit's natural frequency at any given time is unknown a priori, as the RC-WVM sensor size varies as per its intended use. In one embodiment, a typical sensor is qualified for patient IVC diameters nominally in the range of about 14 mm to about 28 mm. This means that overall sensor diameter range will be from somewhat less than about 14 mm to somewhat greater than 28 mm in order to detect changes in IVC dimensions above and below nominal size range. When sensor diameter lies in the lower end of that size range, e.g., below about 19 mm or even below about 15 mm, the amplitude of ring-back signal that may be produced by the sensor will be relatively low due to reduced inductive coupling and therefore can present challenges with respect to detection and accurate signal analysis. A further challenge in determining the proper excitation signal may be imposed by regulatory requirements, which typically require any such signal to have a limited bandwidth and power. These challenges can be met in a number of ways.

In one embodiment, the excitation signal provided by signal generation module 20a and delivered by antenna module 16 may be configured as a pre-defined transmit pulse (e.g. a single frequency burst) to energize the RC-WVM sensor. In this embodiment, the transmit pulse frequency is chosen to optimally energize the sensor on the assumption the sensor is in the lower diameter range as the smaller sensor diameter produces a lower ring-back signal amplitude. In one alternative, the transmit pulse frequency may be chosen on the assumption that the sensor is at its smallest diameter, which would have the lowest ring-back signal amplitude, thus requiring optimal excitation to ensure the ring-back signal is at a sufficiently detectable level to obtain reliable readings. The same pre-defined transmit pulse frequency is used to energize the sensor for the duration of the signal measurement, e.g., 60 seconds. However, when the vessel expands, the optimal excitation frequency changes and amplitude of the ring-back signal may decrease resulting in less reliable readings being taken.

In another embodiment, a frequency sweep function may be used to more reliably transmit the excitation signal at or close to the optimal frequency. In one example, the signal generation module 20a performs a frequency sweep function by sequentially outputting a preestablished number of transmit pulses at pre-defined frequencies over a range of expected implant natural frequencies (in one example, five transmit pulses are used). The ring-back sensor signals captured during the frequency sweep function are processed through receiver-amplifier module 20b, communications and data acquisition module 22 and optionally external devices 18. All ring-back signals (corresponding to the preestablished number of transmit pulses) are received and processed. Of the resonant frequencies detected out of the preestablished number of transmit pulses sent, the one with the highest amplitude is chosen as the optimal transmit frequency. The optimal excitation frequency is then used as the excitation transmit pulse to energize the sensor for the duration of the signal measurement, e.g., 60 seconds. Note that depending on the size of the sensor at the time of the transmit pulse sweep, all ring-back signals from the preestablished number of transmit pulses may be detected and any used as the optimal resonant frequency.

In the frequency sweep method explained above, the system selects the frequency with highest amplitude as detected during the execution of the frequency sweep function. As explained, the amplitude of the resonant frequency produced is dependent on IVC dimension (e.g., area or diameter) at the monitoring location, with larger dimensions resulting in larger signal amplitude. Employing this methodology, the system may therefore tend to choose excitation frequencies that are more optimal for larger sensor sizes. Subsequently, during signal acquisition, when the dimension of the vessel decreases (e.g. due to respiration collapse), the excitation can become sub-optimal, potentially resulting in low or insufficient signal quality when the vessel collapses. Further alternative excitation frequency determination methods may be utilized to address this.

In one such further alternative embodiment, the excitation frequency is determined using a two-tier approach. Firstly, an initial excitation frequency is determined, using, for example, the frequency sweep function described above. Signal generation module 20a is therefore configured to transmit at the frequency determined by means of the frequency sweep function during an initial observation period, which should be sufficiently long to cover at least one respiration cycle. The sensor resonant frequency is assessed during this period and the highest detected frequency is subsequently chosen as the excitation frequency for the remaining of the signal measurement. This approach may favor the selection of higher frequencies, corresponding smaller sensor areas (which can be the worst case for signal quality), and as such may provide a more reliable excitation.

A limitation of the method described in the preceding paragraph is envisaged when considering a situation of significant collapse of the IVC due to respiration. In this case, as the initial frequency sweep will tend to pick a resonant frequency corresponding to larger sensor/vessel dimension, when the IVC reaches its maximum level of collapse, the resonant frequency of the sensor could deviate significantly from the excitation frequency, resulting in suboptimal excitation. This, coupled to the reduced amplitude of the sensor response (due to small sensor area) can result in unreliable resonant frequency detection (due to low signal quality) and potentially incorrect excitation frequency determination.

In order to overcome this issue, a further refinement may be employed in which the system repeatedly executes the frequency sweep function described above during a period of pre-defined length, which should be sufficiently long to cover at least one respiration cycle. As the excitation frequency sequentially changes between the pre-defined frequencies (including frequencies corresponding to the smallest sensor areas), a more optimal excitation is achieved in situations of large IVC collapse and small sensor. As in the method above, the system picks the highest observed resonant frequency as the excitation frequency for the remaining of the signal measurement.

In another implementation, the frequency of the excitation signal is adjusted dynamically during signal acquisition. In one embodiment, the amplitude or signal-to-noise ratio (SNR) of the response signal from the RC-WVM sensor is monitored, either continuously (for each sample) or periodically. If the signal amplitude is detected to fall below a pre-defined threshold (e.g., due to larger collapse of the IVC), a new frequency sweep (using any of the methods previously described) is executed, allowing re-tuning to the latest sensor resonant frequency.

In a further embodiment, the output frequency of signal generation module 20a is continuously adjusted after each measurement point. In this case, the resonant frequency of the sensor is computed for each acquired sample in between sample acquisitions. The excitation frequency for the next sample is therefore adjusted to the latest measured resonant frequency. Provided that the sampling rate of the system is faster than the dynamics of the IVC collapse, this method will consistently ensure optimal excitation.

Embodiments described above require signal processing algorithms for frequency detection that can be executed in real-time in communications and data acquisition module 22. Fast Fourier Transform (FFT) can be used for said purpose. However, if high resolution of the detected IVC dimension is required, the length of the required FFT could result in prohibitive computational time and would therefore be not suitable to allow frequency determination in between sample acquisitions. Alternatively, a variation of the traditional FFT such as the Zoom FFT can be used. This technique allows analyzing focusing on a given portion of the spectrum reducing this way the length of the FFT and therefore its computational time without compromising resolution of the detected frequency.

Determination of the optimal transmit frequency using any of the methods described above is a key in providing efficient excitation of the RC-WVM sensor, given that the amount of RF power that can be transmitted via antenna 16 will be subject to limits imposed by applicable regulations aimed to ensure efficient use of the frequency spectrum. As an additional means to minimize the level of intentional RF emissions, the dependency between RC-WVM sensor area and strength of the sensor response signal can be considered. As previously stated, larger sensor area will typically result in larger mutual inductance (and therefore magnetic field coupling) between the antenna 16 and the RC-WVM sensor. Taking this into account, signal generation module 20a can be controlled in such a way that the output RF power is adjusted as a function of the output frequency. In particular, maximum power is transmitted when the detected resonant frequency of the sensor is at the high end of the expected sensor bandwidth, which corresponds to the smallest sensor area and therefore weakest response. The output power is therefore monotonically reduced as the frequency decreases, facilitating thus compliance to applicable radio regulations.

In another implementation, the amplitude of the RC-WVM sensor response signal is monitored, and the output of the transmitter is dynamically adjusted, e.g. to achieve a constant signal amplitude (similar to an automatic gain control application). As described in the previous paragraph, this methodology can allow a tighter control of the emitted RF power. In addition, this methodology provides means to ensure the amplitude of the received signal does not cause saturation of the receiver stage, which can otherwise lead to inaccuracies in the signal processing algorithms that are subsequently applied in order to determine the fundamental component of the sensor.

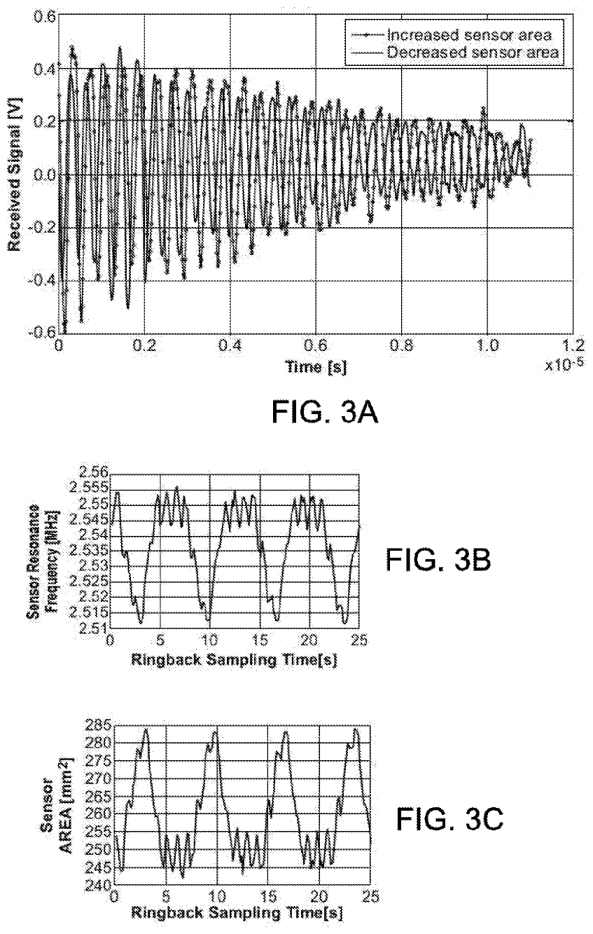

FIGS. 3A, 3B and 3C, respectively, illustrate examples of signals from in vivo tests, respectively, a raw ring-back signal, detection of the resonant frequency and conversion to an IVC dimension using a reference characterization curve. FIG. 3A shows the raw ring-back signal in the time domain with the resonant response of the RC-WVM implant decaying over time. Modulation of the implant geometry due to changes in IVC shape result in a change in the resonant frequency, which can be seen as the difference between the two different plotted traces. FIG. 3B shows the RC-WVM implant signal from FIG. 3A as converted into the frequency domain and plotted over time. The resonant frequency from FIG. 3A is determined (e.g., using fast Fourier transform) and plotted over time. The larger, slower modulation of the signal (i.e., the three broad peaks) indicate the respiration-induced motion of the IVC wall, while the faster, smaller modulation overlaid on this signal indicate motion of the IVC wall in response to the cardiac cycle. FIG. 3C shows the frequency modulation plotted in FIG. 3A converted to a sensor area versus time plot. (Conversion in this case was based on a characterization curve, which was determined through bench testing on a range of sample diameter lumens following standard lab/testing procedures.) FIG. 3C thus shows variations in IVC dimension at the monitoring location in response to the respiration and cardiac cycles.

As will be appreciated by persons of ordinary skill, accurate and reliable interpretation of a complex signal such as shown in FIGS. 3A-C requires good signal fidelity and confidence with respect to both the excitation signal and the ring-back signal from the RC-WVM. Embodiments disclosed herein thus provide solutions to potential problems to help ensure the best possible signal fidelity and confidence.

One way in which signal fidelity can be compromised is when defective hardware within the control system leads to inaccurate readings. A mechanism is thus needed to validate the accuracy of data produced by the system. In one embodiment, data accuracy may be validated by reading a known frequency signal created by signal generation module 20a with receiver-amplifier module 20b and confirming the output of the system matches the known input. Thus, in an embodiment a known, fixed frequency and amplitude signal portion is included within the captured signal to allow for validation of the raw data files off-line. Receiver-amplifier 20b in conjunction with the communications and data acquisition sub-module 22 starts to capture the produced signal as soon as the transmit cycle begins. The transmit signal is large in amplitude and, as such, creates a small leakage signal through the transmit/receive (T/R) switch 92 that reaches the receiver channel. Since the latter has a very large gain, the resultant signal at the receiver's output can be detected and processed in order to determine its frequency, which is known a priori because the transmitter has been programmed to create such a frequency. In another alternative, a known or fixed frequency signal portion may be included in the sensor raw data capture by allowing transmit/receive switch 92 to leak the known excitation signal from the transmit side to the receive side briefly when switching from transmit to receive.

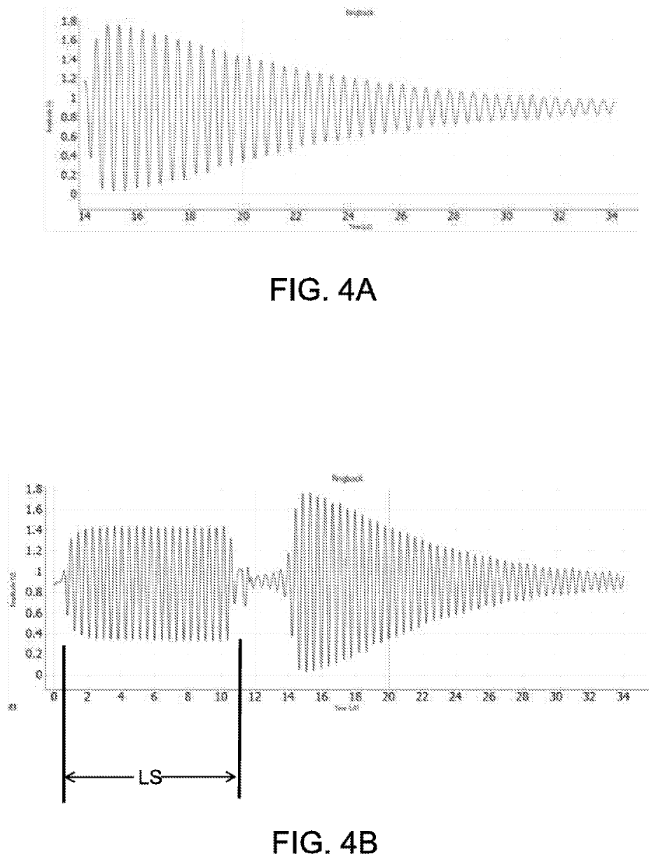

In this manner, when receiver-amplifier module 20b begins to capture the received signal, the first portion of the signal is the known frequency portion. The brief signal leakage is illustrated by comparing FIGS. 4A and 4B. FIG. 4A illustrates a ring-back signal as may be received by the control system after the RC-WVM sensor is energized by a signal from the transmit side in typical operation without any signal leakage through T/R switch 92. The signal in FIG. 4A begins at maximum amplitude at the left side when the RC-WVM coil is first energized and decays over time as energy is dissipated. Note that in this example, the ring-back signal begins at time 14 μs, which represents the time delay for the transmit signal to send and energize the sensor. (The excitation signal is delivered beginning at time 0, which is not shown in FIG. 4A, but is shown in FIG. 4B.) The signal in FIG. 4B shows the received signal when leakage through the switch is permitted as in embodiments described above. The leakage portion of the signal (LS) begins at approximately time zero because there is no delay waiting for the sensor to be energized. Then by limiting the leakage signal (LS) to a time before the sensor ring-back signal is anticipated, the leakage signal does not interfere with readings from the sensor, but at the same time provides a known frequency validation signal that can be checked against the control system output.

In one embodiment, the process of providing a leakage signal as a known frequency hardware validation signal may comprise the following:
1. An RF transmitter outputs a known pulse via an antenna to energize the sensor.
2. A transmit/receive switch is configured to allow signal leakage from the transmit side to the receive side. The receiver electronics begin to capture the receiver data while the transmitter is active.
3. The transmit/receive switch changes the antenna connection fully to the receiver electronics to detect the sensor RF response.
4. The receiver electronics continues to capture the sensor signal via an ADC.
5. The captured ADC data is stored in the microcontroller and sent to the laptop for longer-term storage. The data now includes the transmit portion of the transmit/receive cycle within the data packet. The data packet also includes the frequency programmed into the RF transmitter.
6. Data can then be validated by comparing the frequency and amplitude of the transmit portion of the data signal data against the programmed frequency and pre-defined thresholds for expected amplitude.

A further problem that can be encountered with systems of the type described herein is interference from background noise. Excessive electromagnetic noise or external electromagnetic interference from nearby devices can result in the system detecting a reading that does not relate to the sensor signal. During normal operation, the system attempts to detect a signal elicited by the sensor in response to the excitation signal that is delivered to the sensor during the transmit cycle. A sufficiently strong external signal could couple into the system and mask the sensor signal, potentially resulting in an incorrect measurement.

This problem can be solved according to the present disclosure by providing a mechanism to assess the electromagnetic background noise prior to commencement of the measurement. In one embodiment, the system is operated in normal mode, i.e., the transmit mode is engaged and a known test frequency is transmitted that is sufficiently away from the expected sensor bandwidth/excitation frequency. In this way, the sensor is not energized and hence produces no ring-back signal response. The control system then toggles to receiver mode as in normal operation and any received signal is recorded. Since no response from the sensor is present (because of the “detuned” transmit frequency), the received signal is made up completely of background electromagnetic noise. Appropriate corrections or accommodations in the signal processing can then be employed based on the detected background noise. In one option, the control system assesses the power of the largest component of the background noise signal. The process is repeated a predefined number of times and an average value is obtained for more consistent measures. The computed signal level is then defined as the background noise.

A background noise evaluation process as described above is not limited to prior to commencing sensor signal recording. In other embodiments, a background noise evaluation as described can also be done at different stages or at multiple points of the sensor signal acquisition process in order to mitigate risks associated to intermittent noise sources or increased noise coupling due to patient moving, etc.

Following assessment of the background noise, the sensor signal is identified through a frequency sweep. Once the sensor response signal is detected, its amplitude is assessed and the resulting value is compared to the previously measured background noise amplitude, effectively computing the Signal to Noise Ratio (SNR). A minimum threshold level is established for the SNR. Any SNR that is below this limit indicates that the external interference is high enough to inhibit reliable measures. This can in turn alert the user to change location or remove any potential source of interference to proceed with using the system.

Use of a characterization curve to translate raw signal output of the RC-WVM sensor into physiologically relevant readings on vessel size and size changes is discussed above in connection with FIGS. 3A and 3C. In general, characterization of raw sensor signals to provide physiologically relevant readings useful to a health care provider is understood in the art. However, RC-WVM sensors as described herein can present unique characterization problems because its characteristic inductance intentionally varies by design. Further, inductance and capacitance characteristics defining the resonant circuit vary due to sensor manufacturing variability. To address these challenges in characterization of RC-WVM sensors, a number of new and different approaches may be utilized.

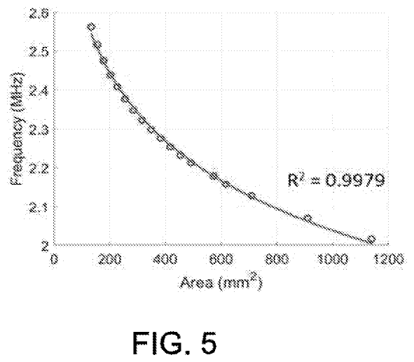

In one embodiment, a sensor characterization curve, such as shown in FIG. 5, is created by sequentially passing the RC-WVM sensor through a series of progressively larger tubes of known area and recording the corresponding frequencies. A unique curve can then be generated from these area-frequency measurements using a number of methods. For example, a curve fitting method can be employed wherein a curve is fit to the raw data by minimizing the error between the fit and the raw data. Curve fitting can be carried out using many different fit types, including, but not limited to, exponential and logarithmic fitting based on the following functions:

Logarithmic: y=c₁·ln(x)+c₂

Exponential: y=c₁·e^{-x/c}^{₂}+c₃

In another example, interpolation may be used wherein a curve is created by interpolating between the recorded area-frequency data. A number of interpolation methods can be used, including a linear interpolation function such as:

$$Linear Interpolation : y = {y}_{1} + ( x - {x}_{1} ) ( \frac{{y}_{2} - {y}_{1}}{{x}_{2} - {x}_{1}} )$$

In addition to the curve type chosen, characterization curves can be generated from individual sensor specific area-frequency data or from the average area-frequency data from a batch of sensors.

Typically, each RC-WVM sensor characterization curve is determined in a clean room during sensor manufacture. However, these curves can shift slightly after the manufacturing and sterilization process. As sensors for clinical use cannot be re-characterized post sterilization, sensor/batch specific manufacturing curves can only be created prior to sterilization. Alternatively, a reference characterization curve can also be generated from independent sensors not for clinical use post sterilization, provided they were manufactured and sterilized in a similar manner to the clinical sensors for which they will be used as a reference.

In a further embodiment, greater characterization accuracy may be achieved as follows. First, during manufacture, area versus frequency data is determined for each sensor. A characterization curve is created from this sensor or batch specific area-frequency data through curve fitting or interpolation as described above before or after sterilization. Then, a sensor measurement is taken, and the result translated into IVC dimension using the characterization curve as created in the preceding step. Measurement error arising from manufacturing variability is thus minimized through the use of sensor or batch specific characterization curves. Using a pre-determined characterization curve allows for more accurate measurements across a larger dimensional range and may avoid the need for in vivo calibration against imaging modalities such as intravascular ultrasound (IVUS), which present other inherent accuracy issues.

Further features, advantages and limitations of embodiments disclosed herein are set out in the following numbered sub-paragraphs:
1. A method and system for validating a sensor signal received from a resonant circuit-based sensor comprising including a known, fixed frequency and amplitude portion signal within an output signal captured from the sensor to allow for validation of the raw data received from the sensor, wherein said validation may optionally be performed off line.
2. A method and system for determining optimal transmit frequency for energizing a resonant circuit sensor, comprising outputting a plurality of pre-defined transmit pulses to energize the sensor over a range of expected sensor frequencies; determining the highest amplitude sensor signal received as corresponding to the optimal excitation frequency; and energizing the sensor at the determined optimal transmit frequency for a duration of a signal measurement, wherein the duration may optionally be about 60 seconds.
3. A method and system for characterizing a dimensionally correlated output signal of the sensor, comprising determining dimension versus frequency data for a sensor during sensor manufacture; creating a characterization curve for the sensor or a batch specific dimension-frequency data through curve fitting or interpolation before or after sterilization of corresponding one or more sensors; taking a measurement with the sensor; translating the sensor result into the desired dimension using the characterization curve as created; minimizing dimension measurement error arising from manufacturing variability through use of sensor or batch specific characterization curves; wherein, optionally, using a pre-determined characterization curve allows for accurate measurements across a large range of dimensions.
4. A method and system for assessing electromagnetic background noise in a sensor system, comprising operating the sensing system in a normal mode, for example with a transmitter engaged, and transmitting a test frequency, said test frequency being sufficiently distant from an expected sensor bandwidth so as to not energize the sensor and elicit a sensor response; toggling the sensor to a receiver mode and recording the received signal with the sensing system, wherein the received signal is made up of background electromagnetic noise; assessing the power of the largest component of this background noise signal; optionally repeating the process a predefined number of times to obtain an average value; and defining the computed signal level is then defined as the background noise.

The foregoing has been a detailed description of illustrative embodiments of the invention. It is noted that in the present specification and claims appended hereto, conjunctive language such as is used in the phrases “at least one of X, Y and Z” and “one or more of X, Y, and Z,” unless specifically stated or indicated otherwise, shall be taken to mean that each item in the conjunctive list can be present in any number exclusive of every other item in the list or in any number in combination with any or all other item(s) in the conjunctive list, each of which may also be present in any number. Applying this general rule, the conjunctive phrases in the foregoing examples in which the conjunctive list consists of X, Y, and Z shall each encompass: one or more of X; one or more of Y; one or more of Z; one or more of X and one or more of Y; one or more of Y and one or more of Z; one or more of X and one or more of Z; and one or more of X, one or more of Y and one or more of Z.

Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A method for controlling a wireless, resonant circuit sensor, the sensor including a variable inductance coil that changes resonant frequency in response to a change in a monitored physical parameter and produces a ring-back signal at a frequency correlated to the physical parameter when energized, the method comprising:
outputting at least one excitation frequency sweep comprising a preestablished number of transmit pulses at pre-defined frequencies over a range of expected implant resonant frequencies;
receiving the ring-back signals for each of the sequentially output transmit pulses;
transmitting at least one initial transmit pulse for a predetermined initial period, wherein the at least one initial transmit pulse comprises one of—
a pulse frequency corresponding to the highest amplitude ring-back signal received from the at least one frequency sweep; or
plural said excitation frequency sweeps;
receiving plural test ring-back signals in response to at least one initial transmit pulse transmitted over the initial period;
identifying an initial ring-back signal corresponding to a preferred excitation pulse frequency; and
selecting said preferred excitation pulse frequency as a measurement transmit pulse frequency;
outputting measurement transmit pulses at the measurement transmit pulse frequency for a subsequent measurement period.

2. The method of claim 1, further comprising:
receiving measurement ring-back signals generated by the sensor in response to the measurement transmit pulses during the measurement period; and
analyzing the measurement ring-back signals to determine a characteristic of the monitored physical parameter.

3. The method of claim 1, wherein:
said transmitting the at least one initial transmit pulse for the initial period comprises—
identifying the ring-back signal with a highest amplitude;
selecting the transmit pulse frequency corresponding to the highest amplitude ring-back signal as the initial transmit pulse frequency; and
transmitting plural initial pulses at the initial pulse frequency; and
said receiving plural initial ring-back signals and said identifying an initial ring-back signal comprise—
exciting the sensor resonant circuit at the initial transmit pulse frequency for the initial period;
receiving the initial ring-back signals from the sensor during the initial period; and
selecting the initial transmit pulse frequency generating the initial ring-back signal with the highest frequency as the measurement transmit pulse frequency.

4. The method of claim 1, wherein:
said transmitting the at least one initial transmit pulse for the initial period comprises outputting repeated excitation frequency sweeps during the initial period; and
said identifying a initial ring-back signal comprises identifying as the preferred excitation pulse frequency a highest observed ring-back signal frequency generated by the repeated excitation frequency sweeps.

5. The method of claim 1, wherein the predetermined initial period comprises a sufficiently long time to encompass at least one respiration cycle.

6. The method of claim 1, further comprising dynamically adjusting the frequency of transmit pulses during acquisition of corresponding ring-back signals.

7. The method of claim 6, wherein said dynamically adjusting comprises:
monitoring at least one of the amplitude or signal-to-noise ratio of the corresponding ring-back signal; and
in response to detection of a ring-back signal amplitude below a pre-defined threshold, outputting a new excitation frequency sweep to identify a new measurement transmit pulse frequency.

8. The method of claim 2, further comprising computing a new measurement pulse signal for each measurement transmit pulse after receiving a measurement ring-back signal during the measurement period.

9. The method of claim 1, further comprising adjusting transmit pulse output power as a function of transmit pulse output frequency.

10. The method of claim 9, wherein said adjusting comprises monotonically reducing transmit pulse output power as transmit pulse frequency decreases.

11. The method of claim 1, further comprising:
monitoring ring-back signals produced by the sensor; and
dynamically adjusting transmit pulse output to achieve a substantially constant ring-back signal amplitude based on the monitored ring-back signals.

12. The method of claim 1, further comprising:
transmitting a known fixed frequency and fixed amplitude signal;
capturing the said known signal as a portion of a captured ring-back signal;
validating signal processing by comparing the captured known signal portion with the transmitted known signal.

13. The method of claim 12, wherein said transmitting and capturing a known signal comprises allowing signal leakage through a transmit/receive switch of a signal generating and receiving control system.

14. The method of claim 1, further comprising assessing electromagnetic background noise prior to outputting the at least one excitation frequency sweep and adjusting signal processing based on a computed background noise signal level.

15. The method of claim 14, wherein said assessing electromagnetic background noise, comprises:
transmitting predetermined a test pulse at a test frequency, wherein said test frequency is selected to be sufficiently distant from an expected sensor excitation frequency so as to not energize the sensor;
receiving a test signal with a sensor ring-back signal receiver, wherein the received test signal is made up of the test pulse and background electromagnetic noise;
defining the background electromagnetic noise based on the received test signal; and
modulating signal processing of the received measurement ring-back signal to eliminate or reduce effects of the defined background electromagnetic noise.

16. The method of claim 1, further comprising:
determining physical parameter versus frequency data for at least one said sensor prior to placement in a patient;
creating a characterization curve for the at least one sensor through curve fitting or interpolation;
taking a measurement with the sensor; and
translating the sensor measurement into a value for the physical parameter using said characterization curve.

17. The method of claim 16, wherein the at least one sensor comprises a sensor batch and the frequency data comprises batch specific parameter-frequency data.

18. The method of claim 16, further comprising minimizing physical parameter measurement error arising from sensor manufacturing variability through use of sensor or sensor batch specific characterization curves.

19. The method of claim 1, wherein the resonant circuit sensor is configured for placement in a patient's vasculature and the physical parameter is a vascular dimension.

20. The method of claim 19, wherein said sensor is specifically configured for placement in a vena cava and the vascular dimension is the area or diameter of the vena cava.

21. The method of claim 20, further comprising correlating the measured area or diameter of the vena cava to patient fluid status.

22. A control system for a wireless, resonant circuit sensor, the sensor including a variable inductance coil that changes resonant frequency in response to a change in a monitored physical parameter and produces a ring-back signal at a frequency correlated to the physical parameter when energized, the control system comprising a transmit/receive switch configured to control signal transmission to and signal receiving from an antenna, a signal generation module configured to generate excitation signals wherein the transmit receive switch controls transmission of the generated signal to the antenna, and a receiver-amplifier module configured to receive and process ring-back-signals received by the antenna and communicated to the receiver-amplifier module by the transmit/receive switch communicating with a processor configured to execute program instructions, wherein the system is configured to:
output at least one excitation frequency sweep comprising a preestablished number of transmit pulses at pre-defined frequencies over a range of expected implant resonant frequencies;
receive the ring-back signals for each of the sequentially output transmit pulses;
transmit at least one initial transmit pulse for a predetermined initial period, wherein the at least one initial transmit pulse comprises one of—
a pulse frequency corresponding to the highest amplitude ring-back signal received from the at least one frequency sweep; or
plural said excitation frequency sweeps;
receive plural test ring-back signals in response to at least one initial transmit pulse transmitted over the initial period;
identify an initial ring-back signal corresponding to a preferred excitation pulse frequency;
select the preferred excitation pulse frequency as a measurement transmit pulse frequency; and
output measurement transmit pulses at the measurement transmit pulse frequency for a subsequent measurement period.

23. The control system of claim 22, wherein the system is configured to receive measurement ring-back signals generated by the sensor in response to the measurement transmit pulses during the measurement period, and analyze the measurement ring-back signals to determine a characteristic of the monitored physical parameter.

24. The control system of claim 22, wherein the system is configured to:
identify the ring-back signal from the at least one frequency sweep with a highest amplitude and select the transmit pulse frequency corresponding to the highest amplitude ring-back signal as the initial transmit pulse frequency;
transmit an excitation signal at the initial transmit pulse frequency for the initial period;
receive the initial ring-back signals from the sensor during the initial period; and
select the initial transmit pulse frequency that generates the initial ring-back signal with the highest frequency as the measurement transmit pulse frequency.

25. The control system of claim 22, wherein the system is configured to transmit the at least one initial transmit pulse for the initial period by outputting repeated excitation frequency sweeps during the initial period; and
identify an initial ring-back signal by identifying as the preferred excitation pulse frequency the highest observed ring-back signal frequency generated by the repeated excitation frequency sweeps.

26. The control system of claim 22, wherein the system is configured to dynamically adjust the frequency of transmit pulses during acquisition of corresponding ring-back signals by monitoring at least one of the amplitude or signal-to-noise ratio of the corresponding ring-back signal, and, in response to detection of a ring-back signal amplitude below a pre-defined threshold, outputting a new excitation frequency sweep to identify a new measurement transmit pulse frequency.

27. The control system of claim 22, wherein the system is configured to adjust the transmit pulse output power as a function of transmit pulse output frequency by monotonically reducing transmit pulse output power as transmit pulse frequency decreases.

28. The control system of claim 22, wherein the system is configured to monitor ring-back signals produced by the sensor, and dynamically adjust the transmit pulse output to achieve a substantially constant ring-back signal amplitude based on the monitored ring-back signals.

29. A method for characterizing a resonant circuit sensor to correlate sensor output to a measured physical parameter, wherein said sensor comprises a variable inductance coil that changes resonant frequency in response to a change in the physical parameter by producing, when energized, a ring-back signal at a frequency correlateable to the physical parameter, the method comprising:
determining physical parameter value versus frequency data over a range of parameter values and frequencies for at least one said sensor prior to placement in a patient; and
creating a characterization curve for the at least one sensor by plotting a curve with said data using curve fitting or interpolation techniques.

30. The method of claim 29, wherein the physical parameter is an internal vascular lumen dimension comprising diameter or area of the lumen, said sensor being implantable within a vascular lumen and expandable and contractable therewith, characterized in that said determining comprises sequentially placing the sensor in a series of progressively larger or smaller tubes of known dimension and recording the corresponding ring-back signal frequencies when energized in each different sized tube.

31. The method of claim 30, further comprising:
during manufacture, determining a vascular dimension vs frequency data set for each sensor in a sensor batch; and
creating the characterization curve from the sensor batch dimension-frequency data through curve fitting or interpolation prior to sterilization of the sensors.

32. The method of claim 30, further comprising:
manufacturing and sterilizing a batch of said sensors;
selecting a group of sensors from the sterilized batch of sensors;
designating the selected group of sensors as sensors not for clinical use; and
conducting said determining step only on the group of sensors designated not for clinical use; and
generating the characterization curve for the batch of sterilized sensors based on the dimension versus frequency data generated with the group of sensors designated not for clinical use.

33. A method for assessing electromagnetic background noise prior to outputting an excitation signal for conducting a measurement with a resonant circuit sensor, wherein said sensor comprises a variable inductance coil that changes resonant frequency in response to a change in a physical parameter by producing, when energized, a ring-back signal at a frequency correlateable to the physical parameter, the method comprising:
transmitting predetermined a test pulse at a test frequency, wherein said test frequency is selected to be sufficiently distant from an expected sensor excitation frequency so as to not energize the sensor;
receiving a test signal with a sensor ring-back signal receiver, wherein the received test signal is made up of the test pulse and background electromagnetic noise;
defining the background electromagnetic noise based on the received test signal as signal components distinct from the known test pulse; and
modulating signal processing of the received measurement ring-back signal to eliminate or reduce effects of the defined background electromagnetic noise.

34. A method for validating a sensor signal in a resonant circuit sensor, wherein said sensor comprises a variable inductance coil that changes resonant frequency in response to a change in a physical parameter by producing, when energized, a ring-back signal at a frequency correlateable to the physical parameter, the method comprising:
transmitting a known fixed frequency and fixed amplitude signal;
capturing the said known signal as a portion of a captured signal including a ring-back signal generated by the sensor;
comparing the captured known signal portion with the transmitted known signal; and
validating the sensor ring-back signal when the captured known signal portion matches the transmitted known signal within predetermined limits.

35. The method of claim 34, wherein said transmitting and capturing a known signal comprises allowing signal leakage through a transmit/receive switch of a signal generating and receiving control system.
